(19) United States
(12) Patent Application Publication
(10) Pub. No.: US 2022/0319648 A1
(43) Pub. Date: Oct. 6, 2022

(54) Communication Application for Care Facilities

(71) Applicant: Rakhi Mobil App LLC, Lewis Center, OH (US)

(72) Inventor: Paul Bergsten, Galena, OH (US)

(21) Appl. No.: 17/702,378

(22) Filed: Mar. 23, 2022

Related U.S. Application Data
(60) Provisional application No. 63/167,755, filed on Mar. 30, 2021.

(51) Int. Cl. G16H 10/60; G16H 40/67; G06F 21/31

(52) U.S. Cl. CPC G16H 10/60; G16H 40/67; G06F 21/31

(57) ABSTRACT

Methods for providing secure access to health related information of a patient or resident at a care environment facility are provided. In one aspect, a method includes receiving a list of residents. The method includes generating a unique token for each authorized recipient correspondingly associated with a resident listed on the list of residents. The method includes receiving a request from a user device to access medical information associated with the resident listed on the list of residents. The method includes determining whether to authenticate the request based on the unique token. The method includes facilitating communication, responsive to authenticating the request, between the user device and an electronic medical records server for transmitting the medical information associated with the resident to the user device. Systems and machine-readable media are also provided.

### CROSS-REFERENCE TO RELATED APPLICATIONS

The present application claims the benefit of priority under 35 U.S.C. § 119 from U.S. Provisional Patent Application Ser. No. 63/167,755 entitled “Communication Application for Nursing Homes,” filed on Mar. 30, 2021, the disclosure of which is hereby incorporated by reference in its entirety for all purposes.

### TECHNICAL FIELD

The present disclosure generally relates to communication systems, and more specifically relates to a communication application for care facilities.

### BACKGROUND

Care environment facilities, such as nursing homes, provide care and assistance to residents of those facilities. In some instances, family members of the residents communicate directly with staff of the care environment facilities to check on the wellbeing of the residents. During normal operating environments, these types of communications between a family member and a staff member of the facility are often communicated in a timely manner. When the care environment facility is under staffed, such as during a pandemic, however, the response to family member inquiries may not be immediately attended to and can be delayed.

For example, during a pandemic, the care environment facility may implement procedures to safe guard the residents such as limiting family members or others from entering the facility for visitation. The family member would need to call into or email the facility to get updates on a resident, but, with the facility staff being preoccupied with urgent tasks, these calls may often not get answered and the family member is left without knowing any information about the resident in question.

While the care environment facility may attempt every reasonable effort, during a pandemic or other similarly difficult time, to provide the requested information to family members, such delays often leave the family members frustrated and seeking other options to get information about the resident that may, in turn, tie up other valuable resources. There is a need to streamline communication between family members of residents at care environment facilities and staff at those facilities.

The description provided in the background section should not be assumed to be prior art merely because it is mentioned in or associated with the background section. The background section may include information that describes one or more aspects of the subject technology.

### SUMMARY

The present disclosure provides systems and methods for securely providing access to health related information of a patient at a care environment facility.

According to certain aspects of the present disclosure, a computer-implemented method is provided. The method includes receiving a list of residents. The method includes generating a unique token for each authorized recipient correspondingly associated with a resident listed on the list of residents. The method includes receiving a request from a user device to access medical information associated with the resident listed on the list of residents. The method includes determining whether to authenticate the request based on the unique token. The method includes facilitating communication, responsive to authenticating the request, between the user device and an electronic medical records server for transmitting the medical information associated with the resident to the user device.

According to other aspects of the present disclosure, a system is provided. The system includes a memory comprising instructions and a processor configured to execute the instructions which, when executed, cause the processor to receive a list of residents. The processor is configured to execute the instructions which, when executed, cause the processor to generate a unique token for each authorized recipient correspondingly associated with a resident listed on the list of residents. The processor is configured to execute the instructions which, when executed, cause the processor to receive a request from a user device to access medical information associated with the resident listed on the list of residents. The processor is configured to execute the instructions which, when executed, cause the processor to determine whether to authenticate the request based on the unique token. The processor is configured to execute the instructions which, when executed, cause the processor to facilitate communication, responsive to authenticating the request, between the user device and an electronic medical records server for transmitting the medical information associated with the resident to the user device.

According to other aspects of the present disclosure, a non-transitory machine-readable storage medium comprising machine-readable instructions for causing a processor to execute a method is provided. The method includes receiving a list of residents. The method includes generating a unique token for each authorized recipient correspondingly associated with a resident listed on the list of residents. The method includes receiving a request from a user device to access medical information associated with the resident listed on the list of residents. The method includes determining whether to authenticate the request based on the unique token. The method includes facilitating communication, responsive to authenticating the request, between the user device and an electronic medical records server for transmitting the medical information associated with the resident to the user device.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. It should be noted that although various aspects may be described herein with reference to particular settings, these are examples only and are not to be considered limiting. The teachings of the present disclosure may be applied to other mobile device environments, including but not limited to healthcare environments, telehealth environments, telemedicine environments, independent and assisted living environments, home environment, and other organizational environments well-known in the industry. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example architecture for securely providing access to health related information of a patient or resident at a care environment facility.

FIG. 2 is a block diagram illustrating an example management server, electronic medical records server, facility device, and user device from the architecture of FIG. 1 according to certain aspects of the disclosure.

FIG. 3 illustrates an example process for using the example management server, electronic medical records server, facility device, and user device of FIG. 2.

FIGS. 4A-4D are example illustrations associated with the example process of FIG. 3.

FIG. 5 is block diagram illustrating an example computer system with which the management server, electronic medical records server, facility device, and user device of FIG. 2 can be implemented.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

The disclosed systems and methods allow family members to securely access health related information of a patient or resident at a care environment facility. In certain aspects, the family member can securely communicate with the patient or resident via videochat, text, voice, or any other well-known communication.

The disclosed system addresses a technical problem tied to computer technology and arising in the realm of communication systems, namely the technical problems of securely and efficiently transmitting health related information. The disclosed system solves this technical problem by providing secure authentication for access to the health related information and by transmitting the information from a secure database to a device, and without storing the information at a remote server.

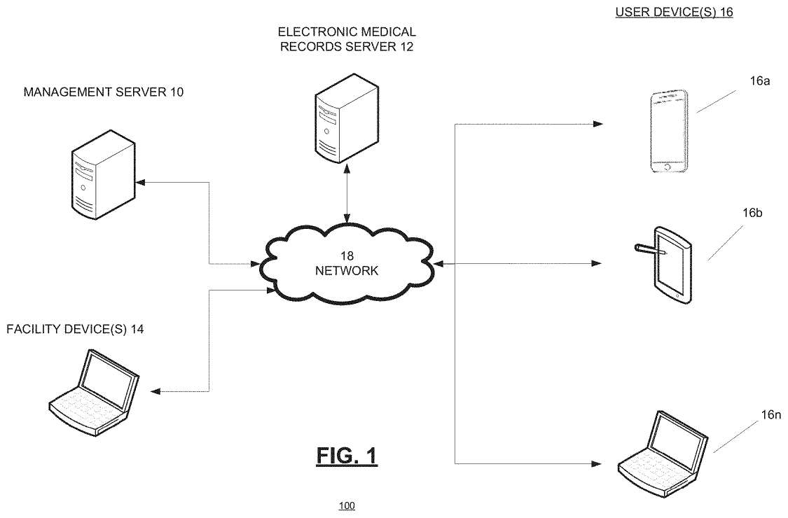

FIG. 1 illustrates an example architecture 100 for securely accessing health related information of a patient. For example, the architecture 100 includes a management server 10, an electronic medical records server 12, at least one facility device 14, and at least one user device 16 (such as, for example, a first user device 16a, a second user device 16b to an nth user device 16n) all connected over a network 18.

The management server 10 can be any device having an appropriate processor, memory, and communications capability for communicating with the electronic medical records server 12, the at least one facility device 14, and the at least one user device 16. For purposes of load balancing, the management server 10 may include multiple servers. The electronic medical records server 12 can be any device having an appropriate processor, memory, and communications capability for communicating with the management server 10, the at least one facility device 14 via the management server 10, and the at least one user device 16. The at least one facility device 14 can be any device having an appropriate processor, memory, and communications capability for communicating with the management server 10, the electronic medical records server 12, and the at least one managed device 16. For example, the at least one facility device 14 can be a tablet computer, a mobile phone, a mobile computer, a laptop computer, a portable media player, an electronic book (eBook) reader, or any other device having appropriate processor, memory, and communications capabilities. The at least one user device 16, such as the first user device 16a, to which the electronic medical records server 12 and the at least one facility device 14 communicate with over the network 18 via the management server 10, can be, for example, a tablet computer, a mobile phone, a mobile computer, a laptop computer, a portable media player, an electronic book (eBook) reader, or any other device having appropriate processor, memory, and communications capabilities. In certain aspects, the management server 10 and the electronic medical records server 12 can be a cloud computing server of an infrastructure-as-a-service (IaaS) and be able to support a platform-as-a-service (PaaS) and software-as-a-service (SaaS) services.

The network 18 can include, for example, any one or more of a personal area network (PAN), a local area network (LAN), a campus area network (CAN), a metropolitan area network (MAN), a wide area network (WAN), a broadband network (BBN), the Internet, and the like. Further, the network 18 can include, but is not limited to, any one or more of the following network topologies, including a bus network, a star network, a ring network, a mesh network, a star-bus network, tree or hierarchical network, and the like.

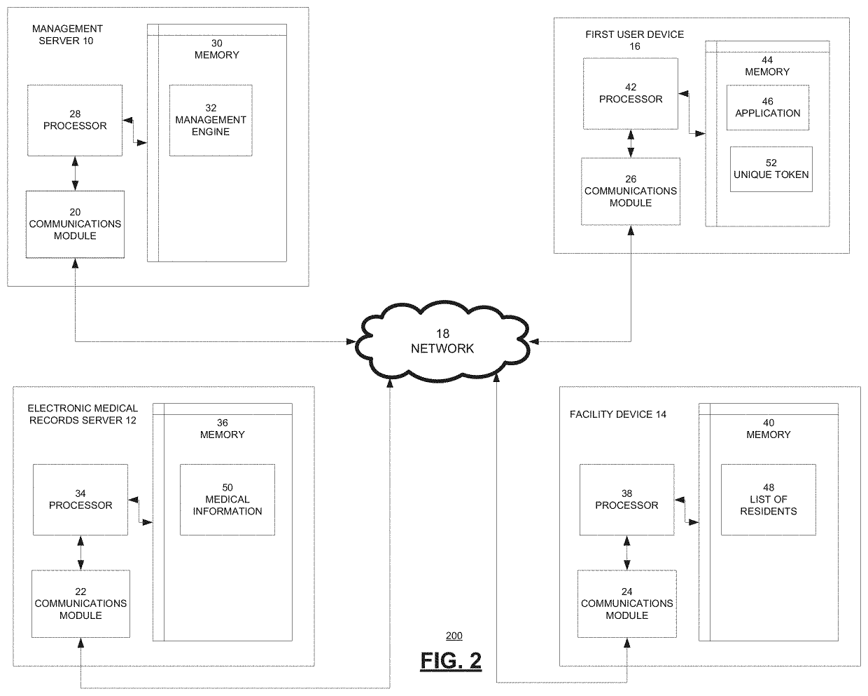

FIG. 2 is a block diagram illustrating examples of the management server 10, the electronic medical records server 12, the at least one facility device 14, and the at least one user device 16, such as the first user device 16a, in the architecture of FIG. 1 according to certain aspects of the disclosure.

The management server 10, the electronic medical records server 12, the at least one facility device 14, and the at least one user device 16, such as the first user device 16a, are connected over the network 18 via respective communications modules 20, 22, 24, 26. The communications modules 20, 22, 24, 26 are configured to interface with the network 18 to send and receive information, such as data, requests, responses, and commands to other devices on the network 18. The communications modules 20, 22, 24, 26 can be, for example, modems or Ethernet cards.

The management server 10 includes a processor 28, the communications module 20, and a memory 30 that includes a management engine 32. The processor 28 of the management server 10 is configured to execute instructions, such as instructions physically coded into the processor 28, instructions received from software in the memory 30, or a combination of both. The management server 10 may correspond to hardware and/or software that implement mobile device management functions. While the management server 10 is illustrated as hosting the management engine 32, it should be understood that the management engine 32 could be hosted on a separate server that communicates with the management server 10.

The electronic medical records server 12 includes a processor 34, the communications module 22, and a memory 36. The processor 34 of the electronic medical records server 12 is configured to execute instructions, such as instructions physically coded into the processor 34, instructions received from software in the memory 36, or a combination of both. Although the electronic medical records server 12 is illustrated as a single server for ease of discussion, it should be understood that the teachings of the present disclosure apply to multiple electronic medical records servers and that each electronic medical records server may be associated with a separate entity.

The at least one facility device 14 includes a processor 38, the communications module 24, and a memory 40. The processor 38 of the at least one facility device 14 is configured to execute instructions, such as instructions physically coded into the processor 38, instructions received from software in the memory 40, or a combination of both.

The first user device 16a includes a processor 42, the communications module 26, and a memory 44. The processor 42 of the first user device 16a is configured to execute instructions, such as instructions physically coded into the processor 42, instructions received from software in memory 44, or a combination of both. The memory 44 of the first managed device 16a may include an application 46.

It should be noted that although various embodiments may be described herein with reference to care environments, this is for example only and not to be considered limiting. The teachings of the present disclosure may be applied to care environments, including but not limited to nursing homes, hospitals, long term care facilities, and to any other environments well-known in the industry.

In certain aspects, the processor 28 of the management server 10, via the management engine 32, is configured to receive a list of residents 48 from the facility device 14. The list of residents 48 includes names of residents who have provided consent for a facility associated with the facility device 14 to disclose medical information 50 to an authorized recipient, such as a user of the first user device 16a. The authorized recipient can be, but is not limited to a family member, a close friend, and a care giver. Each resident on the list of residents 48 consents to allowing his or her medical information 50 to be shared with the authorized recipient. In certain aspects, each of the residents can consent to allowing multiple authorized recipients access to his or her medical information 50. The processor 28 of the management server 10 is configured to assign a unique token 52 specifically to the authorized recipient. The unique token 52 is also associated with a particular resident on the list of residents 48 and will be utilized to securely authenticate that the authorized recipient is allowed to access the medical information 50 of that particular resident. The medical information 50 is stored in the memory 36 of the electronic medical records server 12. In certain aspects, the medical information 50 is stored in a remote database that communicates with the electronic medical records server 12.

In certain aspects, the processor 28 of the management server 10 is configured to receive a request from the first user device 16, via the application 46, to access the medical information 50 associated with the resident associated with the unique token 52 who is listed on the list of residents 48. In certain aspects, after the processor 28 of the management server 10 receives and authenticates the unique token 52 associated with the first user device 16a and the resident, the management server 10 facilitates transmission of the medical information 50 associated with the resident from the electronic medical records server 12 to the application 46 on the first user device 16a. In such aspects, the processor 28 of the management server 10 proxies communication between the electronic medical records server 12 and the first user device 16a and does not store the medical information 50 on the management server 10.

In certain aspects, the processor 28 of the management server 10 is configured to facilitate communication between the first user device 16a and the at least one facility device 14. Such communication between the first user device 16a and the at least one facility device 14 can be, but is not limited to, text, voice, videochat, email, and any other form of communication well-known in the industry. In certain aspects, the processor 28 of the management server 10 is configured to facilitate, via the application 46 of the first user device 16a, further functionalities related to the resident who provided consent such as, but not limited to, telemedicine, business related functions, and other care related functions well-known in the industry. The processor 28 of the management server 10 is configured to transmit notifications to the first user device 16a, via the application 46, to notify the authorized recipient (e.g., a family member or friend) about information associated with the resident.

In certain aspects, the processor 42 of the first user device 16a, via the application 46, is configured to receive further information associated with the resident who provided consent, which may be part of the medical information 50 in some instances, and display such information such as, but not limited to, vitals information, meal intake, personal notes including specific requests, therapy schedules, doctor appoint schedules, resident medications, billing information, insurance information, admission paperwork, significant change notifications, payment transfers, and other health related information well-known in the industry. In certain aspects, the processor 42 of the first user device 16a, via the application 46, is configured to provide further functionalities related to the resident who provided consent such as, but not limited to, telemedicine, business related functions, and other care related functions well-known in the industry. In certain aspects, the processor 42 of the first user device 16a, via the application 46, is configured to receive notifications related to information associated with the resident.

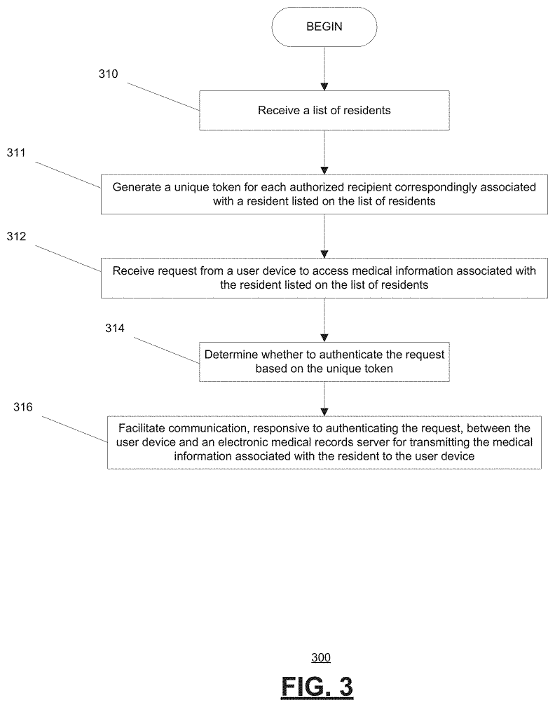

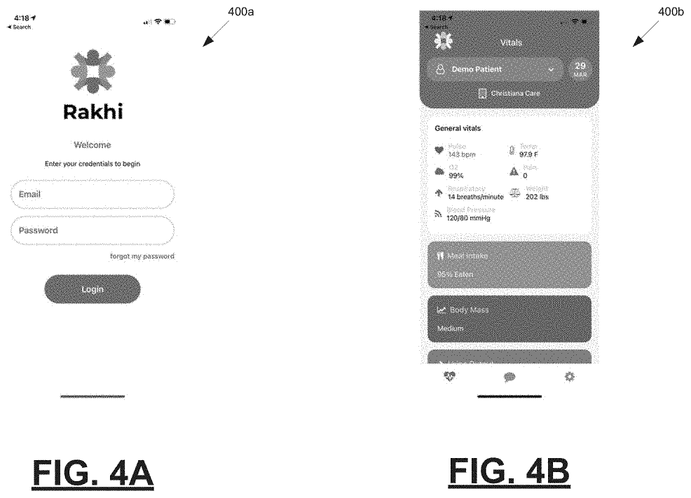

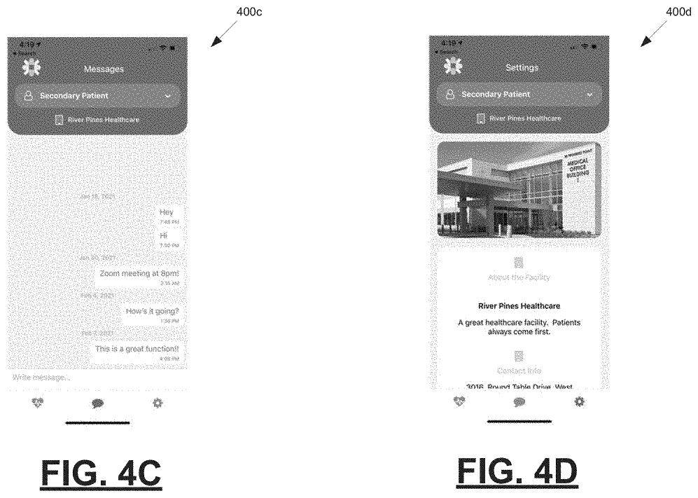

FIG. 3 illustrates an example process 300 for providing secure access to health related information of a patient or resident at a care environment facility using the management server 10, the electronic medical records server 12, the at least one facility device 14, and the at least one user device 16, such as the first user device 16a. While FIG. 3 is described with reference to FIG. 2, it should be understood that the process steps of FIG. 3 may be performed by other systems. Example screenshots 400a-400d of the application 46 are illustrated in FIGS. 4A-4D, respectively.

The process begins by proceeding to step 310 when the processor 28 of the management server 10 receives the list of residents 48 from the at least one facility device 14. At step 311, the processor 28 of the management server 10 generates a unique token 52 for each authorized recipient correspondingly associated with a resident listed on the list of residents 48. As depicted at step 312, the processor 28 of the management server 10 receives a request from a user device, such as the first user device 16a, to access medical information associated with the resident listed on the list of residents 48 who consented that the user associated with the first user device 16a and the unique token 52 be allowed to access associated medical information such as medical information 50. At step 314, the processor 28 of the management server 10 determines whether to authenticate the request based on the unique token 52 associated with the user. As depicted in step 316, in response to authenticating the request, the processor 28 of the management server 10 facilitates communication between the first user device 16a and the electronic medical records server 12 for transmitting the medical information 50 that is associated with the resident to the first user device 16a.

An example will now be described using the example process 300 of FIG. 3. To begin, a resident at a care environment facility consents to allow disclosure of health related information, such as the medical information 50, to an authorized recipient (e.g., family member or friend), such as a user of the first user device 16a. The processor 28 of the management server 10 will receive the list of residents 48, which includes the names of residents who have consented to disclosure of their health related information to authorized family members or friends.

In certain aspects, the processor 28 of the management server 10 also receives a list of authorized family members or friends that includes information detailing a resident and the corresponding authorized family member or friend for comparison with the list of residents 48 to ascertain the authorized recipient who corresponds to the resident. In certain aspects, the list of residents 48 includes information associating the authorized recipients for each resident. Based on the authorized recipient being associated with a particular resident, the processor 28 of the management server 10 generates a unique token 52 for that authorized recipient such that the authorized recipient will be able to access, upon authentication, the medical information 50 associated with that particular resident. Such an authorized family member or friend can download the application 46 onto their device, such as the first user device 16a, and login to the application 46, as exemplarily illustrated in screenshot 400a of FIG. 4A. The authorized family member or friend is associated with the unique token 52 such that when the family member or friend logins into the application 46, the processor 28 of the management server 10 receives the login request and authenticates the request and the unique token 52. Responsive to positively authenticating the request and unique token 52, the processor 28 of the management server 10 facilitates communication between the first user device 16a and the electronic medical records server 12 for transmitting the medical information 50 associated with the resident, from the electronic medical records server 12 to the first user device 16a. The application 46 on the first user device 16a receives and displays the medical information 50, as exemplarily illustrated in screenshot 400b of FIG. 4B.

The processor 28 of the management server 10, via the application 46, also allows the family member or friend associated with the first user device 16a to communicate with at least one facility device 14 via text, email, or videochat, for example, to receive real time information about the resident, as exemplarily illustrated in screenshot 400c of FIG. 4C. The processor 28 of the management server 10 can also transmit notifications to the first user device 16a, via the application 46, to notify the family member or friend about information related to the resident. The family member or friend can also view the facility name and contact info on the first user device 16a via the application 46, as exemplarily illustrated in screenshot 400d of FIG. 4D.

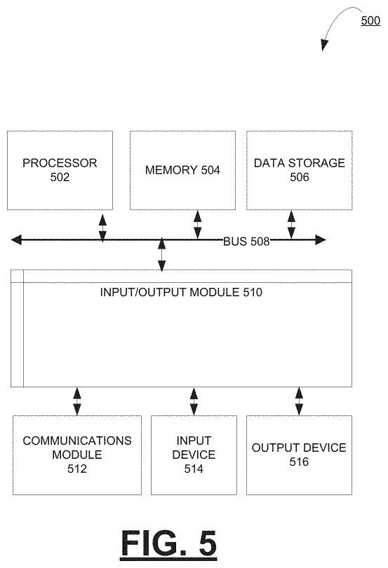

FIG. 5 is a block diagram illustrating an example computer system 500 with which the management server 10, the electronic medical records server 12, the at least one facility device 14, and the at least one user device 16, such as the first user device 16a, of FIG. 2 can be implemented. In certain aspects, the computer system 500 may be implemented using hardware or a combination of software and hardware, either in a dedicated server, or integrated into another entity, or distributed across multiple entities.

Computer system 500 (e.g., the management server 10, the electronic medical records server 12, the at least one facility device 14, and the at least one user device 16, such as the first user device 16a) includes a bus 508 or other communication mechanism for communicating information, and a processor 502 (e.g., the processor 28, 34, 38, 42) coupled with bus 508 for processing information. According to one aspect, the computer system 500 can be a cloud computing server of an IaaS that is able to support PaaS and SaaS services.

Computer system 500 can include, in addition to hardware, code that creates an execution environment for the computer program in question, e.g., code that constitutes processor firmware, a protocol stack, a database management system, an operating system, or a combination of one or more of them stored in an included memory 504 (e.g., the memory 30, 36, 40, 44), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 508 for storing information and instructions to be executed by processor 502. The processor 502 and the memory 504 can be supplemented by, or incorporated in, special purpose logic circuitry.

The instructions may be stored in the memory 504 and implemented in one or more computer program products, e.g., one or more modules of computer program instructions encoded on a computer readable medium for execution by, or to control the operation of, the computer system 500.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 500 further includes a data storage device 506 such as a magnetic disk or optical disk, coupled to bus 508 for storing information and instructions. Computer system 500 may be coupled via input/output module 510 to various devices. The input/output module 510 can be any input/output module. Example input/output modules 510 include data ports such as USB ports. In addition, input/output module 510 may be provided in communication with processor 502, so as to enable near area communication of computer system 500 with other devices. The input/output module 510 may provide, for example, for wired communication in some implementations, or for wireless communication in other implementations, and multiple interfaces may also be used. The input/output module 510 is configured to connect to a communications module 512. Example communications modules 512 (e.g., the communications module 20, 22, 24, 26) include networking interface cards, such as Ethernet cards and modems.

In certain aspects, the input/output module 510 is configured to connect to a plurality of devices, such as an input device 514 and/or an output device 516. Example input devices 514 include a keyboard and a pointing device, e.g., a mouse or a trackball, by which a user can provide input to the computer system 500. Other kinds of input devices 514 can be used to provide for interaction with a user as well, such as a tactile input device, visual input device, audio input device, or brain-computer interface device.

According to one aspect of the present disclosure the management server 10, the electronic medical records server 12, the at least one facility device 14, and the at least one user device 16, such as the first user device 16a, can be implemented using a computer system 500 in response to processor 502 executing one or more sequences of one or more instructions contained in memory 504. Such instructions may be read into memory 504 from another machine-readable medium, such as data storage device 506. Execution of the sequences of instructions contained in main memory 504 causes processor 502 to perform the process steps described herein. One or more processors in a multi-processing arrangement may also be employed to execute the sequences of instructions contained in memory 504. Processor 502 may process the executable instructions and/or data structures by remotely accessing the computer program product, for example by downloading the executable instructions and/or data structures from a remote server through communications module 512 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

The term “machine-readable storage medium” or “computer-readable medium” as used herein refers to any medium or media that participates in providing instructions or data to processor 502 for execution. The term “storage medium” as used herein refers to any non-transitory media that store data and/or instructions that cause a machine to operate in a specific fashion. Such a medium may take many forms, including, but not limited to, non-volatile media, volatile media, and transmission media.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 508. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

In one aspect, a method may be an operation, an instruction, or a function and vice versa. In one aspect, a clause or a claim may be amended to include some or all of the words (e.g., instructions, operations, functions, or components) recited in other one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for”.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A computer-implemented method for providing secure access to health related information of a patient or resident at a care environment facility, the method comprising:
receiving a list of residents;
generating a unique token for each authorized recipient correspondingly associated with a resident listed on the list of residents;
receiving a request from a user device to access medical information associated with the resident listed on the list of residents;
determining whether to authenticate the request based on the unique token; and
facilitating communication, responsive to authenticating the request, between the user device and an electronic medical records server for transmitting the medical information associated with the resident to the user device.

2. The computer-implemented method of claim 1, further comprising:
displaying, via an application on the user device, the medical information associated with the resident.

3. The computer-implemented method of claim 1, further comprising:
facilitating communication between the user device and a facility device associated with the care environment facility.

4. The computer-implemented method of claim 3, wherein the communication comprises one of text, voice, videochat, and email.

5. The computer-implemented method of claim 2, further comprising:
facilitating, via the application on the user device, telemedicine functionalities.

6. The computer-implemented method of claim 1, further comprising:
transmitting notifications to the user device about information associated with the resident.

7. The computer-implemented method of claim 1, wherein the medical information transmitted from the electronic medical records server to the user device is not stored on a server facilitating the communication therebetween.

8. A system for providing secure access to health related information of a patient or resident at a care environment facility, the system comprising:
a memory comprising instructions; and
a processor configured to execute the instructions which, when executed, cause the processor to:
receive a list of residents;
generate a unique token for each authorized recipient correspondingly associated with a resident listed on the list of residents;
receive a request from a user device to access medical information associated with the resident listed on the list of residents;
determining whether to authenticate the request based on the unique token; and
facilitate communication, responsive to authenticating the request, between the user device and an electronic medical records server for transmitting the medical information associated with the resident to the user device.

9. The system of claim 8, wherein the processor is configured to execute the instructions which, when executed, cause the processor to:
display, via an application on the user device, the medical information associated with the resident.

10. The system of claim 8, wherein the processor is configured to execute the instructions which, when executed, cause the processor to:
facilitate communication between the user device and a facility device associated with the care environment facility.

11. The system of claim 10, wherein the communication comprises one of text, voice, videochat, and email.

12. The system of claim 9, wherein the processor is configured to execute the instructions which, when executed, cause the processor to:
facilitate, via the application on the user device, telemedicine functionalities.

13. The system of claim 8, wherein the processor is configured to execute the instructions which, when executed, cause the processor to:
transmit notifications to the user device about information associated with the resident.

14. The system of claim 8, wherein the medical information transmitted from the electronic medical records server to the user device is not stored on a server facilitating the communication therebetween.

15. A non-transitory machine-readable storage medium comprising machine-readable instructions for causing a processor to execute a method, the method comprising:
receiving a list of residents;
generating a unique token for each authorized recipient correspondingly associated with a resident listed on the list of residents;
receiving a request from a user device to access medical information associated with the resident listed on the list of residents;
determining whether to authenticate the request based on the unique token; and
facilitating communication, responsive to authenticating the request, between the user device and an electronic medical records server for transmitting the medical information associated with the resident to the user device.

16. The non-transitory machine-readable storage medium of claim 15, further comprising displaying, via an application on the user device, the medical information associated with the resident.

17. The non-transitory machine-readable storage medium of claim 15, further comprising facilitating communication between the user device and a facility device associated with the care environment facility.

18. The non-transitory machine-readable storage medium of claim 16, further comprising facilitating, via the application on the user device, telemedicine functionalities.

19. The non-transitory machine-readable storage medium of claim 15, further comprising transmitting notifications to the user device about information associated with the resident.

20. The non-transitory machine-readable storage medium of claim 15, wherein the medical information transmitted from the electronic medical records server to the user device is not stored on a server facilitating the communication therebetween.
